(19) United States
(12) Patent Application Publication
(10) Pub. No.: US 2022/0112453 A1
(43) Pub. Date: Apr. 14, 2022

(54) CELL LYSIS WITH A MICROBEAD AND THERMAL RESISTOR

(71) Applicant: Hewlett-Packard Development Company, L.P., Spring, TX (US)

(72) Inventors: Pavel Kornilovich, Convallis, OR (US); Alexander Govyadinov, Convallis, OR (US); Jared Johnson, Convallis, OR (US)

(73) Assignee: Hewlett-Packard Development Company, L.P., Spring, TX (US)

(21) Appl. No.: 17/417,538

(22) PCT Filed: Apr. 30, 2019
(86) PCT No.: PCT/US2019/029800

§ 371 Date: Jun. 23, 2021

(51) Int. Cl. C12N 1/06; C12N 15/10; B01L 3/00

(52) U.S. Cl. CPC C12N 1/066; C12N 15/1017; B01L 3/502761; B01L 2200/0647; B01L 2400/0487; B01L 2300/0663; B01L 2300/14; B01L 2300/1827; B01L 2400/0442; B01L 2200/143

(57) ABSTRACT

Examples herein involve cell lysis with a microbead and thermal resistor. An example apparatus includes a microfluidic channel to pass a volume including a microbead and a biologic sample having nucleic acids enclosed within a cellular membrane. A first thermal resistor may be disposed within the microfluidic channel to move the biologic sample through the microfluidic channel and lyse the cellular membranes in the biologic sample to release the nucleic acids. A microfilter disposed within the microfluidic channel may filter the microbead from the biologic sample and permit the nucleic acids to pass through the filter.

### BACKGROUND

Microfluidics has wide ranging application to numerous disciplines such as engineering, chemistry, biochemistry, biotechnology, and so on. Microfluidics can involve the manipulation and control of small volumes of fluid within various systems and devices such as inkjet printheads, lab-on-chip devices, and other types of microfluidic devices.

### BRIEF DESCRIPTION OF FIGURES

Various examples may be more completely understood in consideration of the following detailed description in connection with the accompanying drawings, in which:

FIG. 1A shows a sectional view of an example apparatus for cell lysis with a microbead and thermal resistor, consistent with the present disclosure;

FIG. 1B shows a sectional view of the example apparatus of FIG. 1A with cell sample and microbeads loaded, consistent with the present disclosure;

FIG. 1C shows a sectional view of the example apparatus of FIG. 1B with a resistor actuated, consistent with the present disclosure;

FIG. 2A shows a sectional view of an example apparatus with a microfluidic channel and a first resistor actuated, consistent with the present disclosure;

FIG. 2B shows a sectional view of the example apparatus of FIG. 2A with a second resistor actuated, consistent with the present disclosure;

FIG. 3A shows a sectional view of an example apparatus for cell lysis with a microbead and with a first resistor actuated, consistent with the present disclosure;

FIG. 3B shows a sectional view of the example apparatus of FIG. 3A with a second resistor actuated, consistent with the present disclosure;

FIGS. 4, 5, 6, 7, 8, 9, 10, 11, and 12 show sectional views of example apparatuses for cell lysis with a microbead and thermal resistor, consistent with the present disclosure;

FIGS. 13, 14, 15, and 16 show sectional views of example apparatuses for cell lysis with a microbead and thermal resistor, consistent with the present disclosure; and

FIG. 17 shows a top view of an example apparatus for cell lysis with a microbead and thermal resistor, consistent with the present disclosure.

While various examples discussed herein are amenable to modifications and alter forms, aspects thereof have been shown by way of example in the drawings and will be described in detail. It should be understood, however, that the intention is not to limit the disclosure to the particular examples described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving lysing cellular membranes. In certain implementations, aspects of the present disclosure have been shown to be beneficial when used in the context of Polymerase Chain Reaction (PCR). While not necessarily so limited, various aspects may be appreciated through the following discussion of non-limiting examples which use exemplary contexts.

Aspects of various examples disclosed herein are directed to an apparatus for cellular lysis. In such examples, the apparatus includes a microfluidic channel to pass a volume including a microbead and a biologic sample having nucleic acids enclosed within a cellular membrane. The apparatus further includes a first thermal resistor disposed within the microfluidic channel to move through the microfluidic channel and lyse the cellular membranes in the biologic sample to release the nucleic acids therein. A microfilter disposed within the microfluidic channel filters the microbead from the biologic sample and permits the nucleic acids to pass through the filter.

Additional examples disclosed herein are directed to an apparatus including a microfluidic channel, a first bubble-driven inertial micropump, a microfilter, and a second bubble-driven inertial micropump. The microfluidic channel may pass a biologic sample for amplification of nucleic acids included in the biologic sample to a microfluidic reaction chamber, and the first bubble-driven inertial micropump disposed within the microfluidic channel may move a volume including the biologic sample and a plurality of microbeads through the microfluidic channel. The microfilter disposed within the microfluidic channel may filter the microbeads from the biologic sample and permit the nucleic acids to pass through the filter to the microfluidic reaction chamber. A second bubble-driven inertial micropump disposed within the microfluidic channel on a side of the microfilter opposite of the first bubble-driven inertial micropump and within a threshold distance of a fluidic reservoir, may move a volume including the nucleic acids and cellular material from the biologic sample through the microfluidic channel.

Yet further examples disclosed herein are directed to a method for lysing cellular membranes. According to such examples, a biologic sample including nucleic acids and a plurality of microbeads may be received at a first end of a microfluidic channel. A first bubble-driven inertial micropump disposed within the microfluidic channel and on a first side of a microfilter, may be activated to agitate a volume including the biologic sample and the microbeads to lyse cellular membranes in the biologic sample and release the nucleic acids therein. The microbeads may be filtered, using the microfilter, from the volume, and a second bubble-driven inertial micropump disposed within the microfluidic channel and on a second side of the microfilter opposite of the first side may be activated to generate a counter flow and remove the microbeads and cellular debris from the microfilter.

Accordingly, in the following description, various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or example may be combined with features of another figure or example even though the combination is not explicitly shown or explicitly described as a combination.

Cell lysis refers to or includes a process of rupturing cell membranes and extracting intracellular components, including deoxyribonucleic acid (DNA) and ribonucleic acid (RNA), among others. Cell lysis has many different applications. Cell lysis may be a step in preparing a sample for polymerase chain reaction (PCR), for example. Some samples may be easier than others to lyse. As a non-limiting illustration, gram-negative bacteria may be lysed more easily using thermal or chemical means, whereas gram-positive bacteria, eukaryotes, and tissue samples, for example, may be harder to lyse using thermal or chemical means.

A microfluidic device may be used to help detect pathogens in the human body and diagnose an illness in a patient. A microfluidic device such as a microfluidic diagnostic chip (MDC) may receive a fluid including an analyte, or sample, and analyze it for purposes of attempting to diagnose a disease in a patient, immunology analysis, and molecular diagnosis, for example.

Microfluidic devices may include inertial pumps to actively move fluids through the microfluidic channels. An inertial pump may include a fluid actuator such as a piezoelectric element or a thermal resistor. The fluid actuator may displace fluid by moving a piezoelectric element or boiling the fluid to form a vapor bubble.

The present disclosure relates to an improved system and method for lysing cells. Particularly, the present disclosure relates to a system and method of conducting mechanical lysis of cells in a microfluidic device. More particularly, the present disclosure relates to a system and method of conducting mechanical lysis of cells in a microfluidic device for amplification of nucleic acids, such as in PCR.

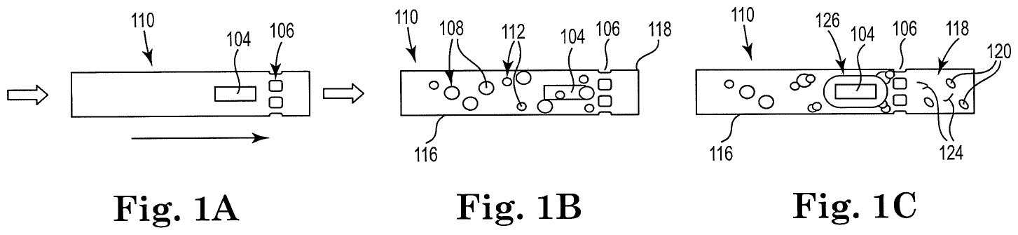

Turning now to the figures, FIG. 1A shows a sectional view of an example apparatus for cell lysis with a microbead and thermal resistor, consistent with the present disclosure. Particularly, apparatus 100 includes a microfluidic channel 110 to pass a volume including a microbead and a biologic sample having nucleic acids enclosed within a cellular membrane. The apparatus 100 includes a first thermal resistor 104 disposed within the microfluidic channel 110 to move the biologic sample through the microfluidic channel 110 and lyse the cellular membranes in the biologic sample to release the nucleic acids therein. While the thermal resistors described herein may move the biologic sample through the microfluidic channel in the form of a fluid flow, examples are not so limited. For instance, in various examples the thermal resistors may move the biologic sample by agitating the fluid within the microfluidic channel and without creating a fluid flow. As discussed further herein, the operation of the resistor(s) may be impacted by the placement of the resistor within the microfluidic channel and/or relative to other components, such as a fluid reservoir and/or a filter. Yet further, the apparatus 100 includes a microfilter 106 disposed within the microfluidic channel 110 to filter the microbead from the biologic sample and permit the nucleic acids to pass through the filter.

In various examples, the apparatus 100 includes a thermal resistor 104, a microfilter 106, and a microbead (or plurality of microbeads) 108 disposed within a microfluidic channel 110 to lyse cells 112. It is noted that examples of cells 112 are specifically illustrated in FIG. 1B. However, for the sake of clarity, example cells 112 may not be specifically illustrated in other example microfluidic subsequent FIGS.

The thermal resistor 104 referred to above with regard to FIG. 1A may alternatively be known herein as an actuator circuit, an actuator, a resistor, a thermal resistor, or a bubble-driven inertial micropump, for example. Depending upon a location of resistor 104 in the microfluidic channel relative to other components (such as a reservoir and/or the microfilter 106), the resistor 104 may serve a different function other than lysing cells. For example, the resistor 104 may act as a pump, moving fluid within the microfluidic channel 110, and/or a cleaner, removing particulate matter from the microfilter 106. An example thermal resistor may be a thermal inkjet (TIJ) resistor.

FIG. 1B shows a sectional view of the example apparatus of FIG. 1A with cell sample and microbeads loaded, consistent with the present disclosure. As illustrated in FIG. 1B, the biologic sample is introduced in the microfluidic channel 110. The cell fluid 116 may include various cells 112 of interest that are to be lysed, such as cells harvested and/or cultured from plants, animals, or bacteria suspended in an appropriate extracellular fluid medium or in a buffer.

FIG. 1C shows a sectional view of the example apparatus of FIG. 1B with a resistor actuated, consistent with the present disclosure. Applying energy to the resistor 104, referred to herein as actuating the resistor, may super heat the resistor 104 and the surrounding fluid, thereby creating a vapor bubble within the microfluidic channel 110. When the energy is removed from the resistor 104, the vapor bubble collapses. During the vapor bubble collapse, a fluidic bubble jet is produced that concentrates the residual kinetic energy of the bubble in a small area that provides extremely high pressure. The high pressure spikes from expanding and collapsing bubbles, which may be up to about 80 bars of pressure during expansion of the bubble (and thousands of bars during collapse of the bubble), may be used to throw the cells 112 and microbeads 108 around the microfluidic channel 110. When the cells 112 get between two microbeads 108 or between a microbead 108 and either the microfluidic channel 110 wall or a portion of the microfilter 106, the cells 112 may be squeezed and ruptured. The resistor 104 may be activated at a frequency that ensures movement of the cells 112 and microbeads 108 by exposing passing cells 112 and microbeads 108 to multiple high pressure spikes from multiple bubble expansion and/or collapse events.

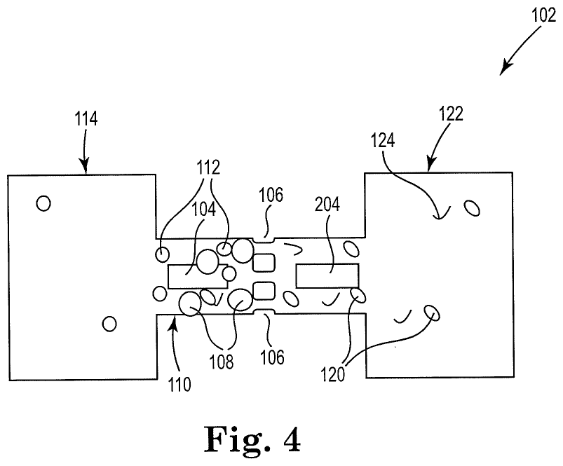

Fluid flow through the microfluidic channel 110, in some examples, may be induced by operation of a single resistor 104 or multiple resistors that may be located within the microfluidic channel 110. Additionally and/or alternatively, the fluid flow may be induced by an external pressure source. Also, the cells 112 and microbeads 108 are thrown around when exposed to multiple pressure spikes from bubble collapse events caused by the resistor 104 within the localized area of the resistor 104. When the cells 112 get between two microbeads 108 or between a microbead 108 and either the microfluidic channel 110 wall or a portion of the microfilter 106, the cells 112 may be squeezed and ruptured, which functions to lyse the cells 112 within the cell fluid 116. Lysate fluid 118 from lysed cells 112 may then be moved through the remainder of the microfluidic channel 110 and into a lysate reservoir 122 (as seen in FIG. 4A).

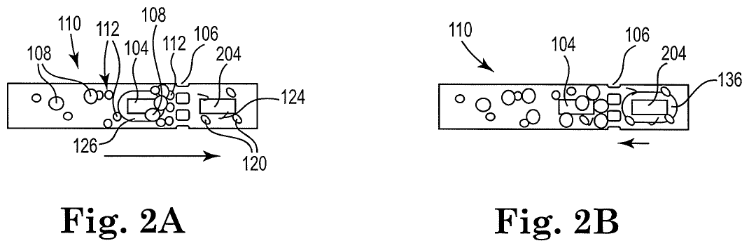

FIG. 2A shows a sectional view of an example apparatus with a microfluidic channel and a first resistor actuated, consistent with the present disclosure. Particularly, FIG. 2A shows a schematic view of a microfluidic channel 110 including two disparate resistors, 104 and 204, disposed within. Each resistor 104, 204 can be individually actuated to induce a different respective flow of fluid within the microfluidic channel 110. For instance, an upstream resistor, such as 104 illustrated in FIGS. 2A and 2B, may induce a flow of the cell fluid 116 and/or may agitate the cell fluid 116. As another illustration, a downstream resistor, such as 204 illustrated in FIGS. 2A and 2B, may induce a counter-flow of the cell fluid 116 and remove particulate matter on the filter 106, thereby performing as a cleaner of filter 106. The arrows show a downstream direction of flow for cell fluid 116 that may be added to the microfluidic channel 110 (shown added in FIG. 2).

A single microfilter, or microscale filter, 106 is shown in the figures. However, it is contemplated that a plurality of microfilters 106 may be included in the microfluidic channel 110. The microfilter 106 may comprise SU8 pillars, epoxy-based negative photoresist pillars, etc., for example, or any other suitable material. A minimal dimension of the microfilter 106, and specifically its holes or openings, may be chosen such as to capture the microbeads 108 and target biological cells (while intact), and to allow lysed cells and inner cell components to pass through the holes or openings.

FIG. 2B shows a sectional view of the example apparatus of FIG. 2A with a second resistor actuated, consistent with the present disclosure. The cell fluid 116 may contain cells 112 to be lysed, microbeads 108, and a lysing buffer. As shown in FIGS. 2A-B in some examples, the micropump 104 may be disposed within the channel 110 in within a threshold distance of the microfilter 106. The intact cells 112 and the microbeads 108 may both be sized such that they both will be trapped by the microfilter 106 (on the upstream side). Alternatively, or additionally, the holes or gaps in microfilter 106 may be sized such that the intact cells 112 and microbeads 108 may not pass through the holes.

The microbeads 108, or microspheres, that may be in or added to the microfluidic channel 110 may include a single microbead or a plurality of microbeads. The microbeads 108 may comprise, for example, glass, silica, alumina, silicon carbide, iron oxide, stainless steel, silica-coated metal, boron nitride, plastic, or other suitable materials. The shape of the beads may be spherical or may not be spherical, such as disk-shaped, rock- or gravel-like, or any other suitable shape. Additionally, the microbeads 108 may be monodispersed or poly-dispersed, for example. The size of the microbeads may vary from a few micrometers to 100 micrometers in diameter, for example. The plurality of microbeads 108 may have a uniform or nearly uniform size, or may vary in size, for example. The number of microbeads 108 that may be added to the sample or cell fluid 116 may vary.

Once the microbeads 108 and the cells 112 are trapped by microfilter 106, the resistor 104 starts firing. Successive and multiple firings of the resistor 104 may also induce fluid flow in the direction shown in FIG. 2A. However, the pressure spikes generated by vapor drive bubbles 126 from multiple, successive firings of the resistor 104 may also start throwing the microbeads 108 and the cells 112 around. The chaotic motion may cause some cells 112 to be squeezed between two microbeads 108, between a single microbead 112 (or more) and an inner wall of the microfluidic channel 110, or between a single microbead (or more) and the microfilter 106. As a result of high shear stress, the cells 112 rupture and release their nucleic acids into solution, making lysate fluid 118. Since nucleic acid fragments 124 are smaller than the gaps in the microfilter 106, the nucleic acid fragments 124 may continue downstream in the system 100 for further processing. Also, a remainder of lysed cells 120 may pass through the microfilter 106.

In various examples, the microfluidic channel 110 may further include sensors. An example sensor may be a flow rate sensor that may measure a flow rate through the microfluidic channel 110. Another example component is a controller circuit, which may be included to adjust a firing frequency of the resistors based on flow rate.

The example apparatus illustrated in FIGS. 2A and 2B may be self-cleaning, including a two resistor arrangement 104, 204. During operation, the microfilter 106 may become clogged by accumulating microbeads 108, cells 112 and/or debris 124 from lysed cells 120, which is shown in FIG. 2A. The second resistor 204 may be placed downstream from the microfilter 106, and may be within a close proximity or within the vicinity of the microfilter 106.

FIG. 2B shows a sectional view of the example apparatus of FIG. 2A with a second resistor actuated, consistent with the present disclosure. Particularly, FIG. 2B shows that as vapor bubbles 136 may be generated by the second resistor 204 there may be a counter-current induced, as indicated by the arrow pointing in an upstream direction, which may apply an upstream pressure toward the microfilter 106. The microbeads 108, cells 112, or cellular debris 124 that may have built up on the upstream side of the microfilter 106 may be shaken loose or cleared from the upstream side of the microfilter 106 by the counter-current.

In the example microfluidic channel 110 shown in FIGS. 2A-B, both resistors 104, 204 may be fired sequentially with the same or different frequencies. Depending on the flow rate and the amount of microbeads 108 in the lysing fluid, each resistor's frequency may also change in time. At a low flow rate, and with a low amount of microbeads 108 in the microfluidic channel 110, both frequencies may be low. The firing frequency of the second resistor 204 may be lower than the firing frequency of the first resistor 104. Moreover, sensors within the microfluidic channel 110, such as upstream and downstream of the microfilter 106 may identify a drop in the flow rate across the microfilter, and adjust the firing rate of the second resistor 204 responsive to a drop in the flow rate above a threshold. The period of firings of both of the resistors 104, 204 may be shorter than average passing time through the resistors 104, 204:

$$\frac{1}{{f}_{TIJ}} < \frac{{L}_{TIJ}}{{\overline{V}}_{flow}} or {f}_{TIJ} < \frac{{\overline{V}}_{flow}}{{L}_{TIJ}}$$

where: f—lysing resistor firing frequency, L_{TIJ}—lysing TIJ resistor length, V_{flow}—is average flow velocity.

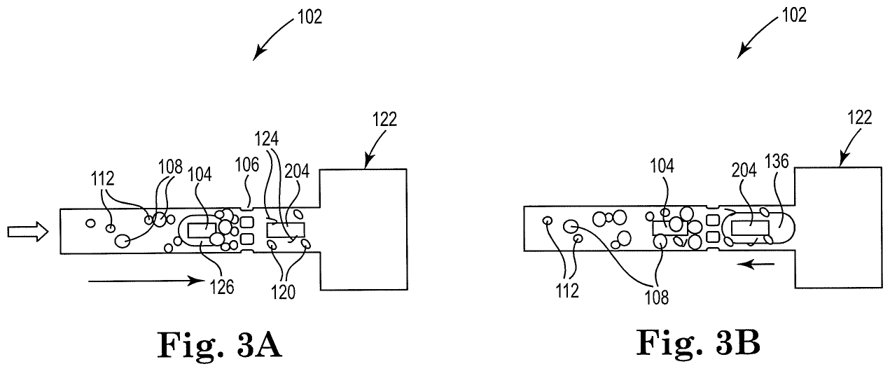

FIG. 3A shows a sectional view of an example apparatus for cell lysis with a microbead and with a first resistor actuated, consistent with the present disclosure. While FIG. 2 illustrates a downstream resistor 204 that agitates the beads near the filter without inducing overall net flow, FIG. 3 illustrates a resistor positioned close to a reservoir to induce a counterflow. In such examples, the downstream resistor 204 not only agitates the particles near the filter but produces a net counterflow from right to left, which is indicated by a directional arrow. The same logic applies to the upstream resistor 104. Particularly, FIG. 3A shows the microfluidic channel 110 before a second resistor 204 is actuated. FIGS. 3A-B show a schematic view of an example microfluidic channel 110 and attached lysate fluid reservoir 122 that may be self-cleaning with counter-flow. The flow direction is shown by the arrow in FIG. 3A, and the counter-flow direction is shown in FIG. 3B by the arrow. The example of FIGS. 3A-B contains the same components as the example of FIGS. 3A-B, such that the description above of the included components also applies to the FIGS. 3A-B example. In addition, the lysate fluid reservoir 122 is shown in the figures in close proximity to the second resistor 204, which may cause counter-flow upstream in the microfluidic channel 110.

FIG. 3B shows a sectional view of the example apparatus of FIG. 3A with a second resistor actuated, consistent with the present disclosure. Particularly, FIG. 3B shows the microfluidic channel 110 after actuation of the second resistor 204. In order to cause the counter-flow, the second resistor 204 may fire and produce vapor bubbles 136, and be disposed within a threshold distance of the lysate fluid reservoir 122. For instance, the second resistor 204 may be located within about 10-15% of the total length of the microfluidic channel 110 away from the lysate fluid reservoir 122. The second resistor 204 may act to clean, clear, or de-clog the microfilter 106, as described above with regards to the example in FIGS. 2A-B.

In various examples, the microfluidic channel 110 may be coupled to a controller that may periodically start the second resistor 204 in order to de-clog the filter 106. Flow-meters or pressure sensors may be added to the microfluidic channel 110 to provide such feedback. A non-limiting example of an integrated, automated lysing system, may include the example microfluidic channel 110 of FIGS. 3A-B, a flow meter, and a controller, for example.

FIG. 4 shows a sectional view of an example apparatus for cell lysis with a microbead and thermal resistor, consistent with the present disclosure. As shown in FIG. 4, a cell fluid reservoir 114 may be connected to the microfluidic channel 110 upstream of the microfilter 106, and a lysate fluid reservoir 122 may be connected to the microfluidic channel 110 downstream of the microfilter 106. The example includes first and second resistors 104, 204 separated by microfilter 106 in the symmetrical microfluidic channel 110. The first resistor 104, which is disposed within a threshold distance of cell fluid reservoir 114, may act as a pump, and may also act to lyse cells 112. The second resistor 204, also disposed within a threshold distance of lysate reservoir 122, may act to de-clog the microfilter 106 as discussed herein.

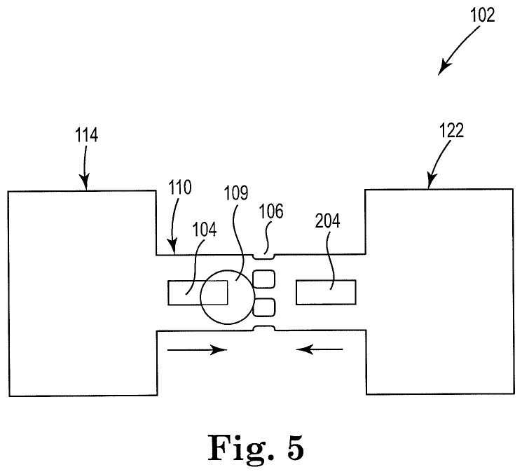

FIG. 5 shows a sectional view of an example apparatus for cell lysis with a microbead and thermal resistor, consistent with the present disclosure. Particularly, FIG. 5 illustrates an example self-cleaning microfluidic channel 110 that includes a single microbead 109, rather than a plurality of microbeads, upstream from the microfilter 106. The single microbead 109 may have a disk-shape, for example, and may comprise SU8, for example. Other shapes, sizes and materials are contemplated for the microbead 109. The example shown includes first and second resistors 104, 204, arranged on first and second sides of the microfilter 106. The first resistor 104, may lyse cells, the second resistor 204 may clean the microfilter 106, such as in the example in FIG. 4 described above. Arrows showing flow (right-pointing arrow) and counter-flow (left-pointing arrow) are included in FIG. 5.

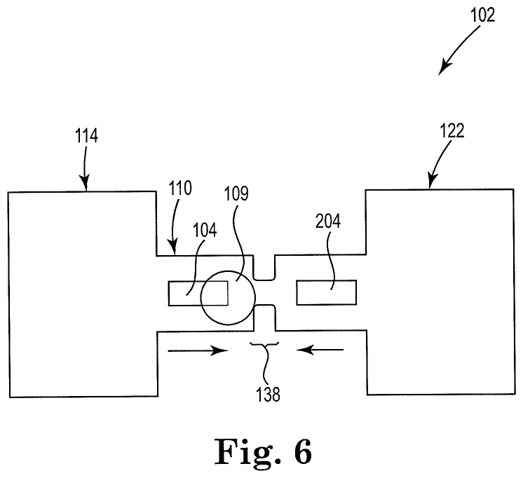

FIG. 6 shows a sectional view of an example apparatus for cell lysis with a microbead and thermal resistor, consistent with the present disclosure. Particularly, FIG. 6 illustrates an example self-cleaning microfluidic channel that may, like the example in FIG. 5, include a single microbead 109. The example may include first and second resistors 104, 204. The apparatus 102, in the example, does not include a microfilter between first and second resistors 104, 204. Device 102 shown includes a pinch point 138 in microfluidic channel 110, which may be a portion of the microfluidic channel 110 with a diameter that may be smaller than the diameter of the remainder of the microfluidic channel 110. The diameter of the pinch point 138 may be smaller in diameter than the diameter of the microbead 109 as well. As such, the microbead 109 may not be able to pass through the pinch point 138, but may be bounced against the upstream side of the pinch point 138 in response to vapor drive bubbles from the first resistor 104. The movement of the hard element 109 may cause lysing of cells caught between the hard element 109 and the pinch point 138, for example, or between the hard element 109 and an inner wall of the microfluidic channel 110. In addition, a narrow channel section, such as pinch point 138, may increase pressure within the narrow section induced by a collapsing bubble generated by resistor 104. The increased pressure within the narrow channel section (or pinch point 138) may provide for faster and more efficient lysing of cells 112 as they pass through the pinch point 138 and are exposed to pressure spikes from collapsing vapor bubbles. In general, narrower channels on one side of the resistor 104, or lysing element, such as at an exit area of the resistor 104, may modify the bubble collapse and increase the bubble pressure.

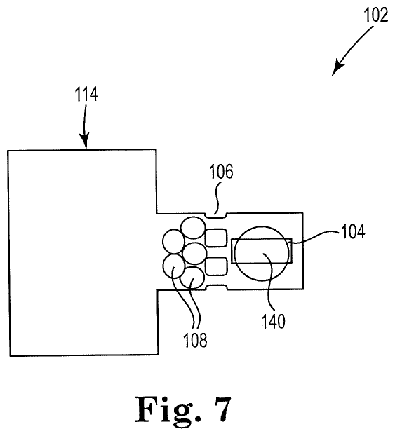

FIG. 7 shows a sectional view of an example apparatus for cell lysis with a microbead and thermal resistor, consistent with the present disclosure. Particularly, FIG. 7 illustrates an example microfluidic channel including a drop ejector. The apparatus 102 includes the cell fluid reservoir 114, and the microfilter 106 may be located in close proximity to the cell fluid reservoir 114 so as to induce a fluid flow through the microfilter 106. Microbeads 108 are shown upstream from the microfilter 106, which may be where lysing takes place. The resistor 104 may be placed downstream of the microfilter 106, and may act to pull (rather than push) on the microbeads 108 and may pull lysate fluid through the microfilter 106. The resistor 104 may act to lyse cells as well as to clean or clear the microfilter 106. An orifice 140 may be located near the resistor 104, through which lysate fluid may be ejected from the device in drops, for further processing. The orifice 140 may be defined by a surface of the microfluidic channel 110, for example.

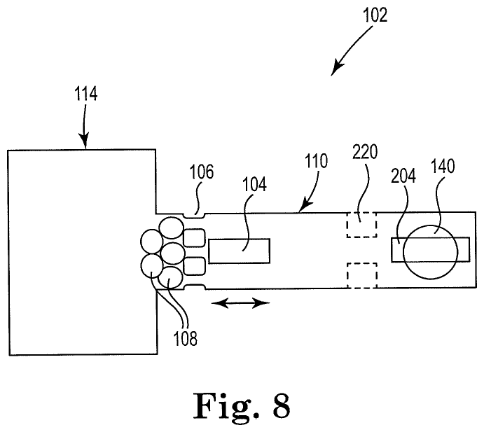

FIG. 8 shows a sectional view of an example apparatus for cell lysis with a microbead and thermal resistor, consistent with the present disclosure. Particularly, FIG. 8 illustrates an example microfluidic channel including a drop ejector. A microfilter 106 may be located in close proximity to the cell fluid reservoir 114 to induce a fluid flow away from inlet reservoir 114. Microbeads 108 are shown upstream from the microfilter 106, which may be where lysing takes place. The second resistor 204 may pull the lysate fluid 118 through the channel and eject the lysate from orifice 140. A narrow channel section 220 may be located downstream from the first resistor 104. The narrow channel section 220 may condense the fluid flow in the microfluidic channel 110, and focus the flow over a second resistor 204. An orifice 140 may be located near the second resistor 204, through which lysate fluid 118 may be ejected from the device in drops, for further processing.

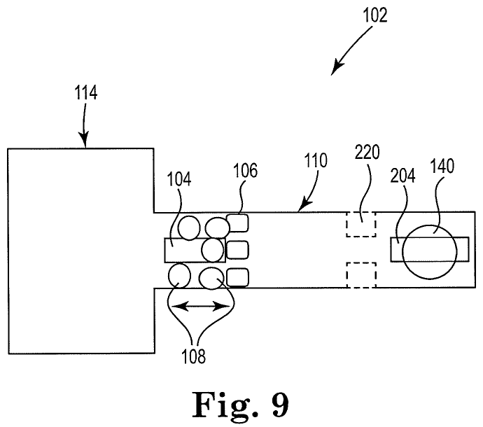

FIG. 9 shows a sectional view of an example apparatus for cell lysis with a microbead and thermal resistor, consistent with the present disclosure. Particularly, FIG. 9 illustrates an example microfluidic channel including a drop ejector. The first resistor 104 may be located in close proximity to the cell fluid reservoir 114 to induce a fluid flow away from inlet reservoir 114, with the microfilter 106 being located downstream from the first micropump 104. Microbeads 108 are shown upstream from the microfilter 106, which may be where lysing takes place. A narrow channel section 220 may be located downstream from the first resistor 104. The narrow channel section 220 may improve efficiency of drop ejection from orifice 140 by focusing the vapor bubble generated by resistor 204 in the z-direction. The second resistor 204 may act to pump lysate fluid 118. An orifice 140 may be located near the second resistor 204, through which lysate fluid 118 may be ejected from the device in drops, for further processing.

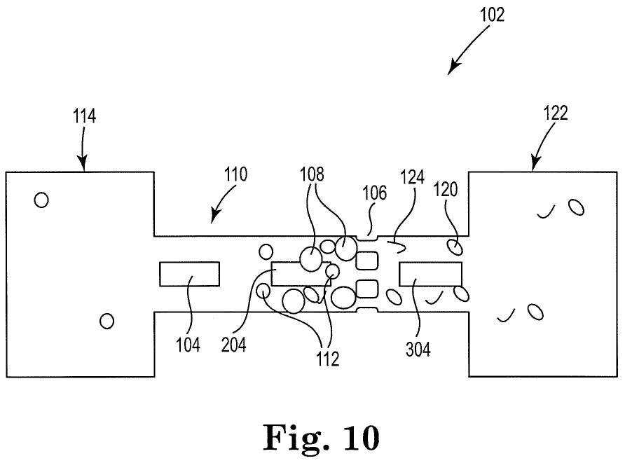

FIG. 10 shows a sectional view of an example apparatus for cell lysis with a microbead and thermal resistor, consistent with the present disclosure. Particularly, FIG. 10 illustrates an example microfluidic channel including more than two resistors. The first resistor 104 may be located in close proximity to the cell fluid reservoir 114 in the microfluidic channel 110 so as to induce a fluid flow in the microfluidic channel 110 away from the inlet fluid reservoir 114. Farther downstream in the microfluidic channel 110 from the first resistor 104 may be located the second resistor 204, which may be in close proximity to the microfilter 106. Microbeads 108 may also be located upstream from the microfilter 106 in order to lyse cells. The second resistor 204 may also act to cause lysis of cells 112 by oscillating the fluid within the microfluidic channel 110. A third resistor 304 may then be located downstream from the microfilter 106 and within a threshold distance of an outlet reservoir 122, so as to induce a counter-flow in the microfluidic channel 110 away from fluid reservoir 122 and clean the microfilter 106.

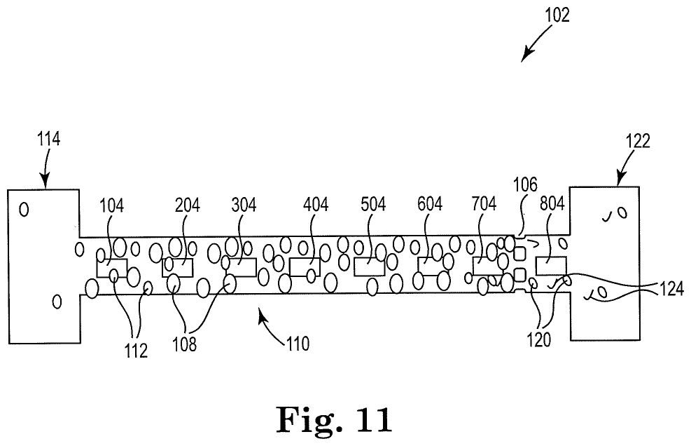

FIG. 11 shows a sectional view of an example apparatus for cell lysis with a microbead and thermal resistor, consistent with the present disclosure. Particularly, FIG. 11 illustrates an example microfluidic channel including an array of resistors located inside and along the length of the microfluidic channel 110, which may individually or collectively act to pump fluid, lyse cells, and/or clean the microfilter 106. The action taken by the individual resistors in the array may depend upon their location in the microfluidic channel 110 of microfluidic device 102, and their proximity to the microfilter 106 and the two fluid reservoirs. For instance, as discussed herein, resistors within a threshold distance of reservoir 114 may act to pump fluid through the microfluidic channel 110, whereas resistors outside of that threshold distance of reservoir 114 may act to agitate and lyse cells. Similarly, resistors located downstream of the microfilter 106 and within a threshold distance of reservoir 122 may act to pull fluid through the channel and to clean the microfilter 106. Although FIG. 11 includes eight resistors, other numbers are also contemplated. Additionally, the resistors may or may not be equally spaced.

A plurality of resistors, such as 104, 204, 304, 404, 504 and 604 in FIG. 11, may, for example, act as pumps and/or agitators to the cell fluid 116, for example. The resistor 704 closest to the microfilter 106, for example, may act to lyse cells 112. Resistor 804 may be located downstream from microfilter 106, and may act to clean the microfilter 106, as described herein above with regards to other examples.

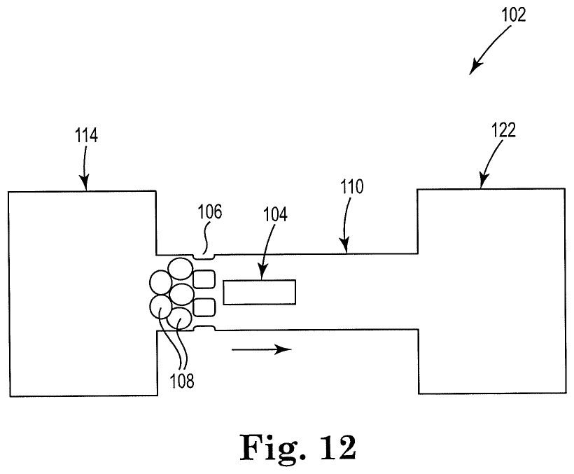

FIG. 12 shows a sectional view of an example apparatus for cell lysis with a microbead and thermal resistor, consistent with the present disclosure. Particularly, FIG. 12 illustrates an example self-cleaning apparatus 102. The microfilter 106 may be located within a threshold distance of the cell fluid reservoir 114. Microbeads 108 are shown upstream from the microfilter 106, which may be where lysing takes place. A resistor 104 located closer to the first reservoir 114 may act as a pump by pushing the microbeads 108 and pull lysate fluid 118 through the microfilter 106. In this example, resistor 104 serves as a push pump by pushing the fluid from left to right, a lysis device by pushing the cells and microbeads against the microfilter, and a filter cleaner. The resistor 104 may also be located within a threshold distance of the second reservoir 122, such that the resistor 104 may act to lyse cells as well as to clean or clear the microfilter 106.

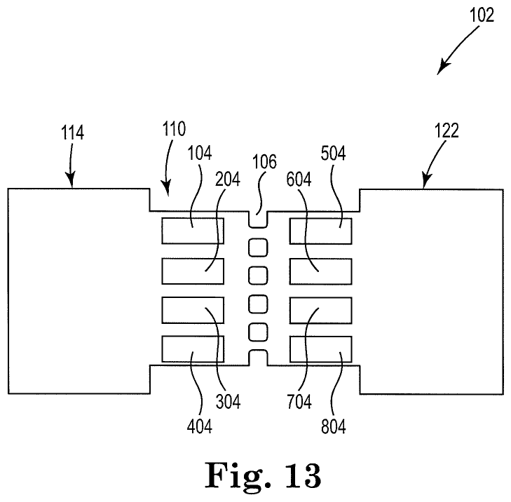

FIG. 13 shows a sectional view of an example apparatus for cell lysis with a microbead and thermal resistor, consistent with the present disclosure. Particularly, FIG. 13 illustrates an example apparatus 102 having a high throughput, or parallel, design. The figure shows a dual-array design, in which a first array of resistors, or micropumps, comprises four resistors 104, 204, 304, 404 that may be arranged vertically within a threshold distance of the cell fluid reservoir 114, and upstream from the microfilter 106 in microfluidic channel 110. A second array of resistors comprises four resistors 504, 604, 704, 804 that may be arranged vertically downstream from the microfilter 106 in the microfluidic channel 110 and within a threshold distance of the lysate fluid reservoir 122. The microfluidic device 102 in the figure is shown having a symmetrical design, although other designs are also contemplated. The first array of resistors 104, 204, 304, 404 may be used to pump and/or lyse cells and the second array of resistors 504, 604, 704, 804 may be used to clean the microfilter 106, for example. In some examples, the first array of resistors may all fire at the same time, and the second array may do the same. However, examples are not so limited. For instance, different ones of the first array and/or the second array may fire at a different time relative to a remainder of the resistors in the array. Similarly, the first array of resistors may fire with a first frequency, and the second array of resistors may fire with a second frequency that is different than the frequency of the first array. Other suitable timing and sequences for firing the resistors are also contemplated. Other suitable numbers of resistors in the arrays are also contemplated. Different resistor sizes are also contemplated.

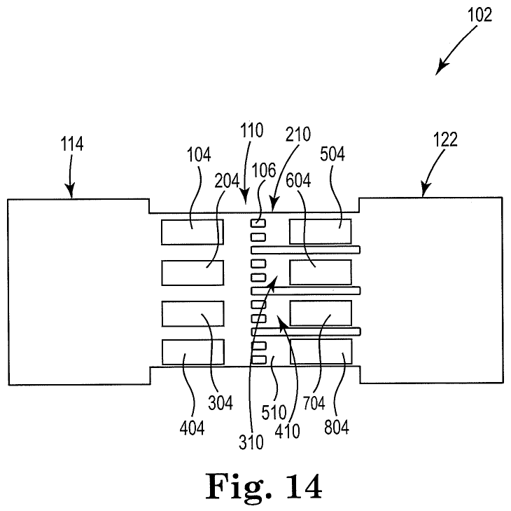

FIG. 14 shows a sectional view of an example apparatus for cell lysis with a microbead and thermal resistor, consistent with the present disclosure. Particularly, FIG. 14 illustrates an apparatus 102 having a high throughput, or parallel, design. The figure shows a dual-array design, in which a first array of resistors, or micropumps, comprises four resistors 104, 204, 304, 404 that may be arranged vertically within a threshold distance of the cell fluid reservoir 114, and upstream from the microfilter 106. A second array of resistors comprises four resistors 504, 604, 704, 804 that may be arranged vertically downstream from the microfilter 106 and within a threshold distance of the lysate fluid reservoir 122. The first array of resistors may be located in the microfluidic channel 110. However, the second array of resistors, as shown, includes each resistor being located in a sub-channel of microfluidic channel 110. The sub-channels are indicted by the numerals 210, 310, 410, and 510.

In FIGS. 13 and 14, dual-array designs are shown. However, it is contemplated that the apparatus 102 disclosed herein may alternatively include a single array or more than two arrays of resistors.

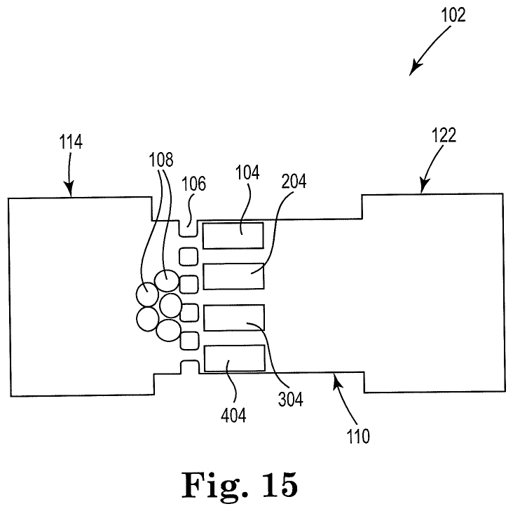

FIG. 15 shows a sectional view of an example apparatus for cell lysis with a microbead and thermal resistor, consistent with the present disclosure. Particularly, FIG. 15 illustrates an example high throughput, or parallel, design. The figure shows a single array design, with the array of resistors 104, 204, 304, 404, arranged vertically and located downstream from, and within a threshold distance of, the microfilter 106 and the reservoir 114. Any suitable number of resistors may be included in the array shown, and the example is not limited to using four resistors.

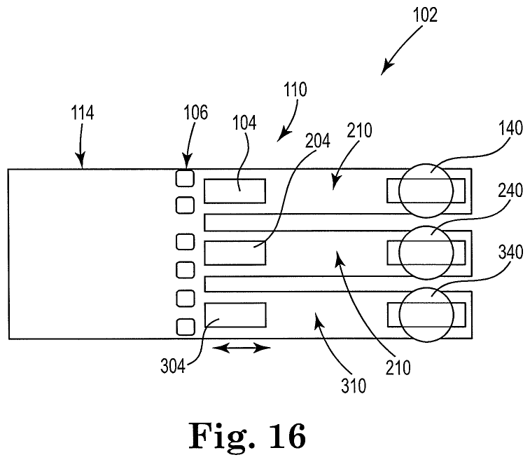

FIG. 16 shows a sectional view of an example apparatus for cell lysis with a microbead and thermal resistor, consistent with the present disclosure. Particularly, FIG. 16 illustrates an example high throughput, or parallel, design. An array of three resistors 104, 204, 304 may be located downstream from the microfilter 106. Although three resistors are shown, other numbers are contemplated by the example. Microfluidic channel 110 is divided into three sub-channels 210, 310, 410, with a single resistor located in each sub-channel. Also, an orifice 140, 240, 340 may be located near the second resistor 204, 304, 404 in each sub-channel 210, 310, 410, respectively. Lysate fluid may be ejected from the apparatus 102 through orifices 140, 240, 340 in drops, for further processing.

The parallel designs shown in FIGS. 13-16 are examples. In alternative example microfluidic mechanical lysis devices, there may be up to dozens or even hundreds of resistors connected or arranged in parallel.

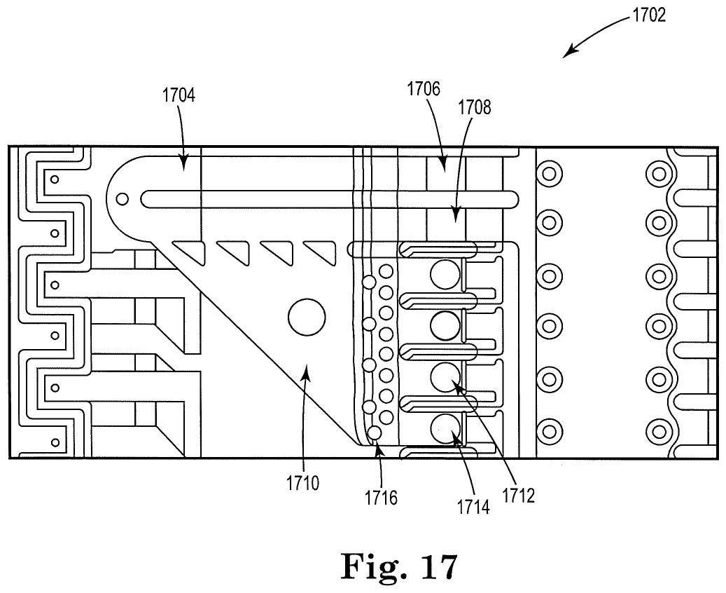

FIG. 17 shows a top view of an example apparatus 1702 for cell lysis with a microbead and thermal resistor, consistent with the present disclosure. A sample, including microbeads and cells, may be recirculated in a recirculation loop indicated as 1704. Two inertial pumps, or resistors, are indicated as 1706, 1708, and may recirculate the sample from loop 1704. A chamber where mechanical lysis may take place is indicated as 1710. Microbeads and cells may be pulled in from the recirculation loop 1704 by ejection pumps 1710, 1712. Ejection pumps 1710, 1712 may include resistors with an orifice disposed nearby, such as may be the case with a thermal inkjet drop ejector. The microbeads may not be able to pass a microfilter 1706 and may be trapped in the chamber 1710. The lysed sample may pass through the microfilter 1706 and to the ejection pumps 1710, 1712. The ejection pumps 1710, 1712 may, in addition to ejecting the lysate, drive vapor bubbles that may generate blowback that may shake the bead/cell mixture that remains inside the lysis chamber 1710. The figure illustrates a single example of the microfluidic device described herein, and other suitable designs and configurations of the microfluidic device are contemplated.

In an example method, microbeads made of a hard material may be added to a lysis buffer. The lysis buffer may then be mixed with a bacterial or tissue sample, for example. The mixture of the lysis buffer and the sample may then be loaded into a microfluidic device, such as into a cell fluid reservoir, as described in the examples disclosed within, for example. A sample, including nucleic acids and a single or plurality of microbeads, may be received at a first end of a microfluidic channel. A first resistor may be disposed within the microfluidic channel and on a first side of a microfilter, such as to agitate a volume including the biological sample and the microbeads to lyse cellular membranes in the biologic sample and release the nucleic acids therein. The microfilter may filter the microbeads from the volume. Lysate fluid, including the nucleic acids, may flow through the microfilter and may be further processed.

In some example methods, a second resistor may be disposed with the microfluidic channel and on a second side of the microfilter opposite the first side to generate a counter-flow and remove the microbeads and cellular debris from the microfilter. In some other examples, the second resistor may be activated to eject lysed cellular membranes and nucleic acids through an orifice defined by a wall of the microfluidic channel. In some examples, a third resistor may be disposed with the microfluidic channel on the first side of the microfilter and within a threshold distance of a fluidic reservoir to move the biological sample toward the first resistor.

The term “sample,” as used herein, generally refers to any biological material, either naturally occurring or scientifically engineered, which contains at least one nucleic acid in addition to other non-nucleic acid material, such as biomolecules (e.g., proteins, polysaccharides, lipids, low molecular weight enzyme inhibitors, oligonucleotides, primers, templates), polyacrylamide, trace metals, organic solvents, etc. Examples of naturally-occurring samples or mixtures include, but are not limited to, whole blood, blood plasma, and other body fluids, as well as tissue cell cultures obtained from humans, plants, or animals. Examples of scientifically-engineered samples or mixtures include, but are not limited to, lysates, nucleic acid samples eluted from agarose and/or polyacrylamide gels, solutions containing multiple species of nucleic acid molecules resulting either from nucleic acid amplification methods, such as PCR amplification or reverse transcription polymerase chain reaction (RT-PCR) amplification, or from RNA or DNA size selection procedures, and solutions resulting from post-sequencing reactions. However, the sample will generally be a biological sample, which may contain any viral or cellular material, including all prokaryotic or eukaryotic cells, viruses, bacteriophages, mycoplasmas, protoplasts, and organelles. Such biological material may thus comprise all types of mammalian and non-mammalian animal cells, plant cells, algae including blue-green algae, fungi, bacteria, protozoa, etc. Representative samples thus include whole blood and blood-derived products such as plasma, serum and buffy coat, urine, feces, cerebrospinal fluid or any other body fluids, tissues, cell cultures, cell suspensions, etc. The sample may comprise a lysate. The sample may also include relatively pure starting material such as a PCR product, or semi-pure preparations obtained by other nucleic acid recovery processes.

In the present specification and in the appended claims, the term “fluid” is meant to be understood broadly as any substance that continually deforms (flows) under an applied shear stress. In one example, a fluid includes an analyte. In another example, a fluid includes a reagent or reactant. In another example, a fluid includes an analyte and a reagent or reactant. In still another example, a fluid includes an analyte, a reagent or reactant, among others. Additionally, in the present specification and in the appended claims the term “analyte” is meant to be understood as any substance within a fluid that may be placed in a MDC. In one example, the analyte may be any constituent substance within a fluid such as, but not limited to, animal or human blood, animal or human urine, animal or human feces, animal or human mucus, animal or human saliva, yeast, or antigens, among others. Further, as used in the present specification and in the appended claims, the term “pathogen” is meant to be understood as any substance that can produce a disease. In one example, the pathogen may be found in any fluid as described above. Still further, in the present specification and in the appended claims the term “reagent” is meant to be understood as a substance or compound that is added to a system in order to bring about a chemical reaction, or added to see if a reaction occurs. A “reactant” is meant to be understood as a substance that is consumed in the course of a chemical reaction.

As used in the specification, the term “cell membrane” refers to or includes any membrane, wall, or other enclosure of a cell.

Terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

The skilled artisan would recognize that various terminology as used in the Specification (including claims) connote a plain meaning in the art unless otherwise indicated. The terms “comprise(s),” “include(s),” “having,” “has,” “can,” “may,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other examples “comprising,” “consisting of” and “consisting essentially of,” the examples or elements presented herein, whether explicitly set forth or not.

As additional examples, the specification describes and/or illustrates aspects useful for implementing the claimed disclosure by way of various structure, such as circuits or circuitry selected or designed to carry out specific acts or functions, as may be recognized in the figures or the related discussion as depicted by or using terms such as blocks, modules, device, system, unit, controller, and/or other examples.

As another example, where the specification may make reference to a “first [type of structure]”, a “second [type of structure]”, etc., where the [type of structure] might be replaced with terms such as circuit, circuitry and others, the adjectives “first” and “second” are not used to connote any description of the structure or to provide any substantive meaning; rather, such adjectives are merely used for English-language antecedence to differentiate one such similarly-named structure from another similarly-named structure designed or coded to perform or carry out the operation associated with the structure (e.g., “first circuit to convert . . . ” is interpreted as “circuit to convert . . . ”).

Based upon the above discussion and illustrations, those skilled in the art will readily recognize that various modifications and changes may be made to the various examples without strictly following the exemplary examples and applications illustrated and described herein. For example, methods as exemplified in the Figures may involve steps carried out in various orders, with one or more aspects of the examples herein retained, or may involve fewer or more steps. Such modifications do not depart from the true spirit and scope of various aspects of the disclosure, including aspects set forth in the claims.

What is claimed is:

1. An apparatus comprising:
a microfluidic channel to pass a volume including a microbead and a biologic sample having nucleic acids enclosed within a cellular membrane;
a first thermal resistor disposed within the microfluidic channel to move through the microfluidic channel and lyse the cellular membranes in the biologic sample to release the nucleic acids therein; and
a microfilter disposed within the microfluidic channel to filter the microbead from the biologic sample and permit the nucleic acids to pass through the filter.

2. The apparatus of claim 1, wherein the microfilter is to filter a plurality of microbeads from lysed cellular membranes and nucleic acids.

3. The apparatus of claim 1, wherein the microbead includes silica, alumina, silicon carbide, stainless steel, boron nitride, glass, or plastic.

4. The apparatus of claim 1, wherein the microfilter includes a plurality of epoxy-based negative photoresist pillars disposed perpendicular to a flow of the biologic sample through the microfluidic channel.

5. The apparatus of claim 1, further including a second thermal resistor disposed within the microfluidic channel on a side of the microfilter opposite of the first thermal resistor.

6. The apparatus of claim 5, further including a flow rate sensor on the side of the microfilter in which the second thermal resistor is disposed, the flow rate sensor to measure a flow rate through the microfluidic channel.

7. An apparatus comprising:
a microfluidic channel to pass a biologic sample for amplification of nucleic acids included in the biologic sample to a microfluidic reaction chamber;
a first thermal resistor disposed within the microfluidic channel to move a volume including the biologic sample and a plurality of microbeads through the microfluidic channel;
a microfilter disposed within the microfluidic channel to filter the microbeads from the biologic sample and permit the nucleic acids to pass through the filter to the microfluidic reaction chamber; and
a second thermal resistor disposed within the microfluidic channel on a side of the microfilter opposite of the first thermal resistor and within a threshold distance of a fluidic reservoir, to move a volume including the nucleic acids and cellular material from the biologic sample through the microfluidic channel.

8. The apparatus of claim 7, further including:
a flow rate sensor to measure a flow rate through the microfluidic channel; and
a controller circuit to adjust a firing frequency of the first thermal resistor and the second thermal resistor based on the flow rate.

9. The apparatus of claim 7, wherein the first thermal resistor includes a thermal resistor to repeatedly generate a vapor bubble and agitate the volume including the biologic sample and microbeads, and the second thermal resistor includes a thermal resistor to create a pressure differential on opposite sides of the microfilter and to move cellular debris from the microfilter to the fluidic reservoir.

10. The apparatus of claim 7, wherein the first thermal resistor includes a thermal resistor to repeatedly generate a vapor bubble and agitate the volume including the biologic sample and microbeads, and the second thermal resistor includes a thermal resistor to create a pressure differential on opposite sides of the microfilter and to push cellular debris away from the microfilter and toward the first thermal resistor.

11. The apparatus of claim 7, further including a second fluidic reservoir disposed within the microfluidic channel on a same side of the microfilter as the first thermal resistor and within a threshold distance of a first thermal resistor, to generate a counter-flow within the microfluidic channel.

12. The apparatus of claim 7, further including a plurality of first thermal resistors disposed within the microfluidic channel on a first side of the microfilter, and a plurality of second thermal resistors disposed within the microfluidic channel on a second side opposite of the first side of the microfilter.

13. A method, comprising:
receiving, at a first end of a microfluidic channel, a biologic sample including nucleic acids and a plurality of microbeads;
activating a first thermal resistor disposed within the microfluidic channel and on a first side of a microfilter, to agitate a volume including the biologic sample and the microbeads to lyse cellular membranes in the biologic sample and release the nucleic acids therein;
filtering, using the microfilter, the microbeads from the volume; and
activating a second thermal resistor disposed within the microfluidic channel and on a second side of the microfilter opposite of the first side to generate a counter flow and remove the microbeads and cellular debris from the microfilter.

14. The method of claim 13, further including activating the second thermal resistor to eject the lysed cellular membranes and nucleic acids through an orifice defined by a surface of the microfluidic channel.

15. The method of claim 13, further including activating a third thermal resistor disposed within the microfluidic channel on the first side of the microfilter and within a threshold distance of a fluidic reservoir to move the biologic sample toward the first thermal resistor.
